(19) United States
(12) Patent Application Publication
(10) Pub. No.: US 2020/0375195 A1
(43) Pub. Date: Dec. 3, 2020

(54) COMPOSITION AND METHOD FOR IMPROVING THE DEVELOPMENT OF PLANTS

(71) Applicant: DANSTAR FERMENT AG, Zug (CH)

(72) Inventors: Mike WHITING, Saskatchewan (CA); Bertrand DELAUNOIS, Toulouse (FR)

(21) Appl. No.: 16/305,703

(22) PCT Filed: Jun. 2, 2017
(86) PCT No.: PCT/EP2017/063438

§ 371 Date: Nov. 29, 2018

(30) Foreign Application Priority Data
Jun. 2, 2016 (FR) 1655009

(51) Int. Cl. A01N 63/20; C12N 1/36

(52) U.S. Cl. CPC A01N 63/20; C12N 1/36

(57) ABSTRACT

Disclosed is a method for increasing the biostimulation efficacy of a live bacterial strain or a composition containing same, the method including a step of inactivating the live bacterial strain, the inactivated bacterial strain obtained in this way having a higher biostimulation efficacy on the development of plants than that obtained with the same bacterial strain when live or with a composition containing same. Also disclosed is an inactivated bacterial strain and a composition including same, for improving the development of a plant.

### TECHNICAL FIELD

The present invention concerns new compositions of inactivated bacteria strains, their preparation method and their use to improve plant development.

### DESCRIPTION OF THE PRIOR ART

The search for new strategies, seeking to maintain or increase the productivity of agricultural systems while reducing the use of chemical inputs, has shown the crucial importance of microorganisms in the biological functioning of agrosystems. These researches have led to the emergence of the concept of “biofertilizers” and “biostimulants”, which can be defined as products containing, for example, inactivated living microorganisms and/or microorganism extracts which, when they are applied onto the soil or the plants, occupy the rhizosphere, even colonizing the plant tissues, and stimulate plant growth by increasing, for example, nutrient assimilation and phytohormone production.

U.S. Pat. No. 5,589,381 describes the isolation of a biological control agent comprising a strain of Bacillus licheniformis, which controls seedling blight due to Fusarium in corn.

U.S. Pat. No. 5,503,652 describes the isolation of strains that can promote root elongation in plants.

U.S. Pat. No. 5,935,839 describes the use of Arthrobacter sp. and Pseudomonas fluorescens for promoting the growth of conifer seedlings, in which plant growth promoting rhizobacteria (PGPR) are selected according to their capacity to grow in the acidic soil and cold typical of conifers.

U.S. Pat. No. 5,503,651 describes the use of PGPR strains that promote the growth of cereals, oilseeds and corn according to chemotactic capacity and by the strains colonizing the roots.

U.S. Pat. No. 5,496,547 teaches the isolation of Pseudomonas mutants that are effective biological agents against Rhizoctonia solani.

U.S. Pat. No. 4,849,008 teaches the application of Pseudomonas onto the roots, plants, seeds, and tuber fragments or soil, of root crops to improve the yield of root crops.

U.S. Pat. No. 4,584,274 describes Pseudomonas strains resistant to bacteriophages useful to promote the growth of root crops.

International application WO2003057861 describes the isolation and identification of a certain number of plant growth promoting rhizobacteria (PGPR), which oxidize sulfur into sulfate usable to promote plant growth such as RAY12, identified as Achromobacter piechaudii; RAY28, identified as Agrobacterium tumefaciens, RAY132, identified as Stenotrophomonas maltophilia; and RAY209, identified as Delftia acidovorans.

U.S. Pat. No. 6,194,193 describes the use of a formulation to enhance plant growth, which comprises a mixture of strains of Bacillus and Paenibacillus, which produce phytohormones.

Another example of known hormonal effect is that of Azospirillum spp (see, in particular, Kucey (1988), Plant growth-altering effects of Azospirillum brasilense and Bacillus C-11-25 on two wheat cultivars. Journal of Applied Microbiology, volume 64, Issue 3, pages 187-196).

Some biofertilizers are composed of living organisms; they must therefore be produced, formulated and sold so that their viability and biological activity are maintained. Furthermore, the success of microbial inoculation for agricultural production is greatly influenced by the number of viable cells introduced into the soil (Duquenne et al., 1999, FEMS Microbiology Ecology 29: 331-339). The viability of the inoculum is an important factor for the success and adequate colonization of the rhizosphere in order to obtain the desired positive effect on plant growth. A major disadvantage of the use of biofertilizers is that the specific soil, temperature and humidity conditions can vary greatly from one site to the other and these variations can influence microbial viability and, consequently, the yield and growth of plants.

There is therefore a need for a composition permitting stimulating plant development with improved efficacy without the disadvantages related to maintaining the viability of this type of biofertilizer/biostimulant.

### SUMMARY

The present invention concerns a method for increasing the biostimulant efficacy of a living bacteria strain or a composition containing it, characterized in that the method comprises a step of inactivating the living bacteria strain, said inactivated bacteria strain thus obtained having a greater biostimulant efficacy on plant development than that obtained with the same strain of living bacteria or with a composition containing it.

The present invention also concerns an inactivated bacteria strain to improve plant development or a composition containing it, characterized in that the inactivated bacteria strain allows improving plant development relative to the same living bacteria strain or the composition containing it.

The present invention also concerns an inactivated bacteria strain to improve plant growth or a composition containing it, characterized in that the inactivated bacteria strain has a greater biostimulant efficacy on plant development than that obtained with the same living bacteria strain or a composition containing it.

The present invention also concerns a method for use or the use of an inactivated bacteria strain or a composition containing it, to improve plant development, characterized in that the inactivated bacteria strain allows improving plant development relative to the same living bacteria strain or the composition containing it.

The present invention also concerns a method for use or the use of an inactivated bacteria strain, or a composition containing it, to improve plant development, characterized in that the inactivated bacteria strain has a greater biostimulant efficacy on plant development than that obtained with the same living bacteria strain or a composition containing it.

Advantageously, the inactivated bacteria strain or composition containing it, obtained according to the method of the present invention, have a greater biostimulant efficacy on plant development than that obtained with the same strain of living bacteria or with a composition containing it.

Advantageously, the composition and method according to the present invention allow improving plant development without having to consider the viability of a bacterial inoculum.

### BRIEF DESCRIPTION OF THE FIGURES

FIG. 1 illustrates the evaluation of the growth of the leaf area of Arabidopsis thaliana as follows: M is culture medium isolated from Delftia acidovorans RAY209; Water is water without active or inactive bacteria or culture medium; B+M is Delftia acidovorans RAY209 in its culture medium; Mp is pasteurized culture medium isolated from Delftia acidovorans RAY209; S is a sulfur suspension; B+water is Delftia acidovorans RAY209 in water; IT45 is a positive control (Bacillus amyloliquefaciens IT45); (B+water)p is a pasteurized solution of Delftia acidovorans RAY209 in water according to the invention.

FIG. 2 illustrates the absorbent root hairs of canola plants (Brassica napus cultivar 5525CL) 36 days after treatment with sterile water.

FIG. 3 illustrates the absorbent root hairs of canola plants (Brassica napus cultivar 5525CL) 36 days after treatment with the culture medium of the strain Delftia acidovorans RAY209.

FIG. 4 illustrates the absorbent root hairs of canola plants (Brassica napus cultivar 5525CL) 36 days after treatment with the strain Delftia acidovorans RAY209 inactivated by treatment with French press (high pressure followed by rapid decompression).

FIG. 5 illustrates the absorbent root hairs of canola plants (Brassica napus cultivar 5525CL) 36 days after treatment with a strain of Lactobacillus rhamnosus inactivated by treatment with French press (high pressure followed by rapid decompression).

FIG. 6 illustrates the absorbent root hairs of canola plants (Brassica napus cultivar 5525CL) 36 days after treatment with the living strain Delftia acidovorans RAY209.

FIG. 7 illustrates the absorbent root hairs of canola plants (Brassica napus cultivar 5525CL) 36 days after treatment with a living strain of Lactobacillus rhamnosus.

FIG. 8 illustrates the absorbent root hairs of canola plants (Brassica napus cultivar 5525CL) 36 days after treatment with the strain Delftia acidovorans RAY209 inactivated by pasteurization.

FIG. 9 illustrates the absorbent root hairs of canola plants (Brassica napus cultivar 5525CL) 36 days after treatment with a strain of Lactobacillus rhamnosus inactivated by pasteurization.

### DETAILED DESCRIPTION

The term “living bacteria” or “living bacteria strains” means bacteria or bacteria preparations with a viability greater than 70%.

The term “inactivated bacteria” or “inactivated bacteria strains” means bacteria or bacteria preparations killed by physical, biochemical, chemical or physicochemical processes and having a viability less than 50%.

The term “biomass” means all the organic and mineral material making up an organism.

The term “biostimulant” means the stimulation of plant development. For example, plant development may include one of the following parameters: rooting, leaf area, flowering, fruiting, plant height, biomass, germination, and harvest yield.

The term “plant growth promoting rhizobacteria” or “PGPR” means rhizosphere bacteria benefiting plant growth and health.

The term “culture medium” means a medium containing the elements necessary to bacteria growth, which permits the culture of bacteria according to the invention. According to one embodiment, the culture medium can contain bacteria according to the invention during their growth or be a culture medium free of the bacteria of the present invention, if these bacteria are separated from their medium by a process implementing, notably but not exclusively, a filtration step or a centrifugation step. According to one embodiment, the culture medium is preferably a liquid medium. All these media, as well as the usual fermentation processes, are well known to the skilled person.

The term “growing medium” means a collection of products intended to serve as growing medium for certain plants. Their implementation leads to the formation of media with water and air porosity, so that they are able to both anchor the absorbing organs of the plants and allow them to be in contact with the solutions necessary for their growth. They are generally composed of organic materials and/or inorganic materials. They are generally composed of peat, other organic materials (in particular coconut fibers, bark, wood fibers, composts) and inorganic materials (in particular soil, sand, pozzolana, clays, mineral wool, perlite, vermiculite).

The present invention concerns a method for increasing the biostimulant efficacy of a living bacteria strain or a composition containing it, characterized in that the method comprises a step of inactivating the living bacteria strain, said inactivated bacteria strain thus obtained having a greater biostimulant efficacy on plant development than that obtained with the same strain of living bacteria or with a composition containing it. In one advantageous embodiment, the inactivation step implemented in the method according to the invention is done by physical, biochemical, chemical or physicochemical processes. In a more advantageous embodiment, the living bacteria strain is inactivated by heat treatment or by high-pressure treatment. Advantageously, the living bacteria strain is inactivated by pasteurization. Even more advantageously, the living bacteria strain is inactivated without its culture medium.

In one particularly advantageous embodiment, the bacteria strain used in the method according to the invention is of the genus Delftia, Achromobacter, Agrobacterium, or Stenotrophomonas. Advantageously, the bacteria strain is of the Delftia genus. More advantageously, the bacteria strain is Delftia acidovorans RAY209 filed with the ATCC on Apr. 25, 2002 under no. PTA-4249, Achromobacter piechaudii RAY12 filed with the American Type Culture Collection (ATCC) on Apr. 16, 2002 under no. PTA-4231, Agrobacterium tumefaciens RAY28 filed with the American Type Culture Collection (ATCC) on Apr. 16, 2002 under no. PTA-4232, or Stenotrophomonas maltophilia RAY 132 filed with the American Type Culture Collection (ATCC) on Apr. 16, 2002 under no. PTA-4233. In a still more advantageous embodiment, the inactivated bacteria strain is the Delftia acidovorans RAY209 strain filed with the ATCC on Apr. 25, 2002 under no. PTA-4249.

The present invention concerns an inactivated bacteria strain, or a composition to improve plant development, characterized in that it comprises at least one inactivated bacteria strain. Advantageously, the present invention also concerns an inactivated bacteria strain to improve plant development characterized in that the inactivated bacteria strain permits improved plant development relative to the same living bacteria strain.

The present invention concerns an inactivated bacteria strain, or a composition to improve plant development, characterized in that it comprises at least one inactivated bacteria strain. Advantageously, the present invention concerns an inactivated bacteria strain to improve plant development characterized in that the inactivated bacteria strain has a greater biostimulant efficacy on plant development than that obtained with the same living bacteria strain or a composition containing it.

The bacteria strain used according to the invention may originate from any bacteria species, in particular bacteria of the genus Delftia, Achromobacter, Agrobacterium, and Stenotrophomonas. According to one advantageous embodiment, the bacteria used according to the invention may originate from the species Delftia acidovorans, Achromobacter piechaudii, Agrobacterium tumefaciens, and Stenotrophomonas maltophilia. More advantageously, the bacteria strain used is chosen from among the group consisting of the strain Achromobacter piechaudii RAY12 filed with the American Type Culture Collection (ATCC) on Apr. 16, 2002 under no. PTA-4231, Agrobacterium tumefaciens RAY28 filed with the American Type Culture Collection (ATCC) on Apr. 16, 2002 under no. PTA-4232, Stenotrophomonas maltophilia RAY 132 filed with the American Type Culture Collection (ATCC) on Apr. 16, 2002 under no. PTA-4233 and/or Delftia acidovorans RAY209 filed with the American Type Culture Collection (ATCC) on Apr. 25, 2002 under no. PTA-4249 in accordance with the Budapest Treaty on the International Recognition of the Deposit of Microorganisms for the Purposes of Patent Procedure.

According to one embodiment, the bacteria strain used in the present invention is a plant growth promoting rhizobacteria or “PGPR”. According to another advantageous embodiment, the inactivated bacteria strain used according to the invention is of the genus Delftia. More particularly, the bacteria strain used according to the invention is the strain Delftia acidovorans RAY209 filed with the American Type Culture Collection (ATCC) on Apr. 25, 2002 under no. PTA-4249 in accordance with the Budapest Treaty on the International Recognition of the Deposit of Microorganisms for the Purposes of Patent Procedure.

According to one advantageous embodiment, the bacteria of the invention are inactivated by physical, chemical, biochemical or physicochemical processes. According to one embodiment, the bacteria of the present invention are inactivated by high pressure treatment (for example using a French press or other methods known in the art). In another embodiment, the bacteria of the present application are inactivated by heat treatment. More advantageously, the bacteria according to the invention are inactivated by pasteurization. According to one particularly advantageous embodiment, pasteurization is conducted by heating the living bacteria to a temperature between 60° C. and 90° C., 62° C. and 88° C., 65° C. and 85° C., 75° C. and 85° C., or 80° C. and 85° C. According to another embodiment, the bacteria according to the invention are pasteurized without their culture medium. According to another embodiment, the bacteria of the present invention are inactivated with their culture medium. According to another embodiment, the bacteria of the present invention are inactivated without their culture medium.

According to one embodiment, the improvement in development and/or growth and productivity of plants and/or the increase in the biostimulant efficacy on plant development notably but not exclusively includes improving one of the following parameters: rooting, leaf area, root development, flowering, fruiting, plant height, biomass, germination, harvest yield, notably in quantity, quality or earlier maturity. According to one embodiment, increasing biostimulant efficacy on plant development includes leaf area, number of absorbing root hairs and/or plant height.

According to one embodiment, the present invention is applicable to all types of plants, in particular but not exclusively to cereal crops (wheat, barley, oats, rye, triticale), root crops (sugar beet, potato, corn), legumes (alfalfa, clover, sainfoin), forage crops (ryegrass, fescue, orchard grass, festulolium, alfalfa, vetch, turnip rape, fodder radish), oilseed crops (soybean, canola, rapeseed, pea, fava bean, white lupine, sunflower), vegetable and market gardening, fruit crops, viticulture and ornamental crops (flower production, lawns, seedling nurseries).

According to one advantageous embodiment, the present invention concerns a composition to improve plant development characterized in that it comprises at least one inactivated bacteria strain, the inactivated bacteria strain having a greater biostimulant efficacy on plant development than that obtained with the same living bacteria strain.

According to one advantageous embodiment, the composition according to the invention comprises 10⁴ CFU/ml to 10¹² CFU/ml, 10⁵ CFU/ml to 10¹² CFU/ml, 10⁶ CFU/ml to 10¹² CFU/ml, 10⁷ CFU/ml to 10¹² CFU/ml, 10⁶ CFU/ml to 10¹¹ CFU/ml, 10⁶ CFU/ml to 10¹⁰ CFU/ml, 10⁶ CFU/ml to 10⁹ CFU/ml, or 10⁶ CFU/ml to 10⁸ CFU/ml bacteria before inactivation.

According to one embodiment, the composition according to the invention comprises at least one strain of inactivated bacteria at 100%, at 99% (with 1% of at least one active bacteria strain), at 98% (with 2% of at least one active bacteria strain), at 97% (with 3% of at least one active bacteria strain), at 96% (with 4% of at least one active bacteria strain), at 95% (with 5% of at least one active bacteria strain), at 94% (with 6% of at least one active bacteria strain), at 93% (with 7% of at least one active bacteria strain), at 92% (with 8% of at least one active bacteria strain), at 91% (with 9% of at least one active bacteria strain), between 90% and 85% (with between 10 and 15% of at least one active bacteria strain), between 85% and 80% (with between 15 and 20% of at least one active bacteria strain), between 80% and 75% (with between 20 and 25% of at least one bacteria strain), between 75% and 70% (with between 25 and 30% of at least one bacteria strain), between 70% and 65% (with between 30 and 35% of at least one active bacteria strain), between 65% and 60% (with between 35% and 40% of at least one active bacteria strain), between 60% and 55% (with between 40 and 45% of at least one active bacteria strain) or between 55% and 50% (with between 45% and 50% of at least one active bacteria strain).

According to one particularly advantageous embodiment, the composition according to the invention comprises at least one inactivated bacteria strain and a vehicle compatible for agriculture. More particularly, the vehicle compatible for agriculture is appropriate for the administration of the inactivated bacteria on the plant and/or on the soil. According to one embodiment, the vehicle is in the solid and/or liquid form. According to another embodiment, the vehicle is water. According to another embodiment, the vehicle is a water-herbicide mix. According to another embodiment, the vehicle is a water-fertilizer mix. According to another embodiment, the vehicle is a culture medium.

According to one particularly advantageous embodiment, the composition according to the invention comprises an inactivated bacteria strain isolated from its culture medium.

According to one advantageous embodiment, the composition comprising at least one inactivated bacteria strain may be in the form of powder, granules, microgranules, seed treatments, liquid formulations, bacteria encapsulation or liquid suspensions. More particularly, the composition according to the invention is in liquid form.

According to one advantageous embodiment, the composition according to the invention is in combination with an appropriate formulation comprising powders, notably wettable powders, granules, microgranules, seed treatments, bacteria encapsulations, liquid formulations, including but not limited to suspensions, in water, in a solvent or in a culture medium.

According to one advantageous embodiment, the composition according to the invention is in an appropriate form for soil treatment, treatment of the root part of the plant, treatment of the leaf part of plant, treatment of the flowering part of the plant, treatment of the fruiting part of the plant and/or treatment of the seed. According to another advantageous embodiment, the composition according to the invention is administered simultaneously or successively, by application to the soil, by root soaking, by treatment of seeds or by incorporation and/or coating with a growing medium, film-coating with plant protection products or fertilizers or any other vehicle or by any means allowing immediate contact or future contact of the composition with the seeds or plants to be inoculated. More particularly, application to the soil is done particularly, but not exclusively, by spraying, spreading, watering, ground treatment, fertigation, drip, in the seedling furrow or in the open.

According to one advantageous embodiment, the composition according to the invention comprises the inactivated bacteria strain in combination with other living microorganisms, inactivated or in extracts, such as bacteria, fungi and/or yeasts. More particularly, the composition according to the invention comprises the inactivated bacteria strain in combination with other inactivated bacteria strains, said bacteria promoting plant development, nutrition and protection.

According to one advantageous embodiment, the composition according to the invention also comprises fertilizers, herbicides, insecticides, fungicides, bactericides, mineral solutions and/or growing media. According to another advantageous embodiment, the composition according to the invention also comprises a substrate. More particularly, the substrate comprises organic material, notably, but not exclusively peat, inorganic materials, notably but not exclusively soil and/or sand and/or clay and/or other soil components and/or synthetic substances. More particularly, the synthetic substance may be an absorbant material such as, for example a granulate material.

According to one embodiment, the composition containing an inactivated bacteria strain or the inactivated bacteria strain of the present invention permits greater plant development improvement than that obtained with the same composition containing the same living bacteria strain, or the same living bacteria strain.

According to another aspect, the present invention concerns the use of an inactivated bacteria strain or a composition comprising it to improve plant development. According to one embodiment, the use of an inactivated bacteria composition or strain of the present invention permits greater plant development improvement than that obtained with the same living bacteria strain or the same composition containing it.

According to another aspect, the present invention concerns a method to improve the development of a plant comprising the administration of an inactivated bacteria composition according to the invention or an inactivated bacteria strain according to the invention. According to one embodiment, the method comprising the administration of an inactivated bacteria strain composition or an inactivated bacteria strain according to the invention permits greater improvement of plant development than that obtained with the same composition containing the same living bacteria strain, or the same living bacteria strain.

According to another aspect, the present invention concerns a plant obtained by using the composition according to the invention or the inactivated bacteria strain according to the invention to improve plant development.

According to another aspect, the present invention concerns a plant obtained by using a method to improve the development of a plant comprising the administration of an inactivated bacteria composition or an inactivated bacteria strain according to the invention.

### EXAMPLES

The present application will be better understood on reading the following examples that are given to illustrate the application and not to limit the scope.

Tests have been done to verify the impact of some strains and mainly living or killed Delftia acidovorans with or without their culture medium, on the development of a model plant.

### Example 1

1-1/ Preparation of Bacterial Strain Inocula: Delftia acidovorans

The desired bacterial concentration in each inocula has been estimated at an equivalent of 4·10¹¹ CFU/m³ with a commercial strain of Delftia acidovorans, referenced LPC8. Since the pots used during the tests have a volume of 0.0003 m³, it was determined that 1.2·10⁸ CFU needed to be introduced per pot.

1-2/ Counting Technique for the Bacterial Suspensions:

The bacterial strain Delftia acidovorans RAY209 is sold in a liquid formula containing the suspension of bacteria in its culture medium (BioBoost Liquid or BBL). In order to know the bacterial concentration of BBL, the bacteria in the product were counted. 1 ml of solution was drawn off from the initial Bag-in-Box™ (BBL) bacterial suspension in order to be placed in a test tube. Peptone water (9 ml) was added to the tube containing 1 ml of bacterial suspension in order to make a 10⁻¹ dilution. A second sample of 1 ml of this dilution was put into a test tube and 9 ml of peptone water were added in order to make a 10⁻² dilution. The process was then repeated identically until a dilution of 10⁻⁴ and 10⁻⁸ was obtained. 100 μl were drawn off from each of these 10⁻⁴ and 10⁻⁸ dilutions to inoculate the surface of a Petri dish (TSA medium: trypto-casein soy agar). A total of 6 Petri dishes were inoculated per dilution and they were incubated for 48 h at 30° C. (ATCC., 2015; Larcher., 2015). The colonies on the surface of the dishes were counted and the concentration in the BBL (commercial product) was estimated at 2·10⁷ CFU/ml.

1-3/ Counting Technique for the Bacterial Suspensions:

The bacterial strain Delftia acidovorans RAY209 is sold in a liquid formula containing the bacteria suspension in its culture medium. Preparation of the bacteria inoculum in the culture medium: “bacteria+supernatant” (B+M):

The BBL bacterial suspension was used as it is for the “bacteria+supernatant” (B+M) protocol.

1-4/ Preparation of the Inoculum: “Bacteria+Water”:

1400 ml of BBL bacterial suspension were centrifuged at 8500 rpm (13000 g) for 15 minutes. The bacterial pellet was dissolved in 700 ml of water and centrifuged again. This step was repeated 3 times in order to eliminate any remaining culture supernatant. The pellet was then resuspended in tap water (around 700 ml). The bacterial concentration was measured again in this solution according to the protocol described in Example 1-2. After counting the colonies on the surface of the dishes, the bacteria concentration was estimated at 4·10⁷ CFU/ml. This suspension as such is the “bacteria+water” protocol;

1-5/ Preparation of the “Bacteria+Water, Pasteurized” Protocol ((B+Water)p):

Approximately 200 ml of the “bacteria+water” suspension described in Example 1-2 were heated in a water bath for 20 minutes at 80° C. in order to obtain the “bacteria+water, pasteurized” ((B+water)p) suspension.

1-6/ Preparation of the “Culture Supernatant” Protocol (M):

The BBL bacterial suspension was centrifuged at 8500 rpm (13000 g) for 15 minutes and approximately 600 ml of supernatant were drawn off for inoculating the plants. This sample is the “culture medium” (M) protocol.

1-7/ Preparation of the “Pasteurized Culture Supernatant” Protocol (Mp):

The BBL bacterial suspension was centrifuged and 200 ml of culture supernatant were recovered after centrifugation in order to be heated in the water bath for 20 minutes at 80° C.

The resulting solution is the “pasteurized culture supernatant” (Mp) protocol.

1-8/ Preparation of the “Sulfur” (S) Protocol:

1 g of sulfur was mixed into 100 ml of water in a container to obtain the “sulfur” (S) protocol.

1-9/ Preparation of the Bacillus amyloliquefaciens IT45 (IT45) Positive Control Protocol:

An atomized microbial preparation of Bacillus amyloliquefaciens IT45, in the powder form and concentrated to 2·10¹⁰ CFU/g, was resuspended in water to obtain the target concentration of 4.10⁷ CFU/ml.

### Example 2

2-1/ Plant Preparation (Arabidopsis thaliana)

The plants were grown in a solid medium under nonsterile conditions in order to approximate natural conditions in the field according to the following repetitions:

Biological repetition=8 protocols×3 repetitions×6 iterations=144 plants

2-2/ Preparation of Seedlings

The seeds were stored at 4° C. in order to ensure correct and synchronized germination (ABRC, 2015). Approximately 300-400 seeds were sparsely placed in 3 Petri dishes (14 cm in diameter) on germinating paper. The equivalent of 5 ml of water were added so as to simply soak the germination paper. The Petri dishes were placed in the refrigerator (4° C.) for 3 days to ensure stratification of the seeds.

2-3/ Preparation of Micro-Greenhouses and Pots

The plants were grown in pots (6×6×7 cm) placed in 6 micro-greenhouses (22×16×18 cm). The pots were filled with 150 g of a breeding ground-sand mixture (⅔ breeding ground and ⅓ sand, m/m).

The micro-greenhouses were covered with irrigation matting on the inside to maintain a moist environment for the plants.

The seeds were then removed from the Petri dishes and placed in pots of soil (5-6 seeds per pot) with tongs. One shoot per pot was retained once the seeds germinated.

2-4/ Plant Inoculation and Growth

Each seedling was inoculated at the time of sowing with the solutions/suspensions described in Example 1 as follows. The seedlings were subjected to a day/night cycle of 16 h of day at 23° C. followed by 8 h of night at 18° C. The pots were watered once or twice a day during the diurnal part of the cycle. The humidity was not regulated and was approximately 70-80%. Each protocol was inoculated according to the following doses:
A) Delftia acidovorans RAY209 in its culture medium (B+M): 6 ml/pot (18 pots)
B) “bacteria+water” (B+water): 3 ml/pot (18 pots)
C) “(bacteria+water) pasteurized” (B+water)p: 3 ml/pot (18 pots)
D) “pasteurized culture supernatant” (Mp): 6 ml/pot (18 pots)
E) water: 3 ml/pot (18 pots)
F) “culture supernatant” (M): 6 ml/pot (18 pots)
G) Suspension of Bacillus amyloliquefaciens IT45: 1 ml/pot (18 pots)
H) Sulfur (S): 0.01 g/pot (18 pots) (1 ml per plant of a 1% (m/v) solution).

The various pot lots were randomized so that the tests were distributed homogeneously in the area used to eliminate variable disparities (temperature, brightness, aeration, humidity, etc.) present in the greenhouse and cultivation room. 3 units of 8 mini-greenhouses were set up in the cultivation room, with rotation within the units.

2-5/ Measurements and Analyses

Plant growth was evaluated by the development of the leaf area over time. The plants were photographed from above every 2 days and each photo was analyzed using Fiji software to be able to calculate the leaf area of each plant throughout the cycle.

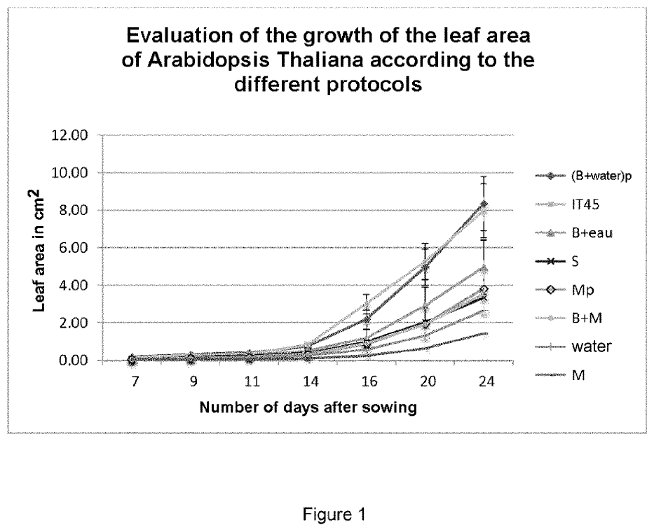

The measurement data were entered into an Excel file (leaf area, dry mass, fresh mass) and these data were analyzed using XLSTAT software. The Tukey's test (post-hoc multiple comparison test) was used to make conclusions on the significant differences between the means of the protocols (see FIG. 1).

TABLE 1
Means of the leaf areas for each protocol and per day

| | Day 7 | Day 9 | Day 11 | Day 14 | Day 16 | Day 20 | Day 24 |
|---|---|---|---|---|---|---|---|
| (B + water)p | 0.179 a | 0.302 a | 0.398 a | 0.742 ab | 2.166 ab | 4.960 a | 8.339 a |
| IT45 | 0.126 ab | 0.242 ab | 0.314 a | 0.837 a | 2.998 a | 5.263 a | 7.961 a |
| B + water | 0.070 bc | 0.149 abc | 0.254 ab | 0.498 abc | 1.167 ab | 2.917 a | 4.975 a |
| S | 0.044 c | 0.125 bc | 0.226 ab | 0.360 abc | 0.964 ab | 2.021 a | 3.326 a |
| Mp | 0.050 c | 0.120 bc | 0.164 ab | 0.315 abc | 0.859 ab | 1.932 a | 3.809 a |
| B + M | 0.056 c | 0.106 bc | 0.158 ab | 0.296 abc | 0.890 ab | 1.913 a | 3.538 a |
| water | 0.053 c | 0.101 bc | 0.173 ab | 0.252 bc | 0.553 ab | 1.312 a | 2.646 a |
| M | 0.018 c | 0.033 c | 0.050 b | 0.098 c | 0.218 b | 0.624 a | 1.414 a |
| Pr > F | 0.000 | 0.001 | 0.010 | 0.004 | 0.025 | 0.033 | 0.036 |
| Significant | Yes | Yes | Yes | Yes | Yes | Yes | Yes |
| (>95%) | | | | | | | |

a, b and c are homogenous groups of treated plants, used for doing the Tukey's statistical test.
Pr > F: threshold value for the degree of significance

The differences observed in the leaf area measurements of each protocol are significant (see Table 1). A significant difference between the positive control (Bacillus amyloliquefaciens IT45) and the negative control (water) was observed. Moreover, the (B+water)p protocol showed the best results in terms of plant leaf area (greater than the positive control) in comparison to other protocols. The culture supernatant M showed the least leaf growth.

### Example 3

Study on the Effect of Inactivation of the Delftia and Lactobacillus Strains on the Growth Parameters of Canola Seedlings

The objective of this study is to determine the effect of pasteurization treatment on the Delftia acidovorans and Lactobacillus rhamnosus strains on the growth of canola seedlings (Brassica napus cultivar 5525CL). More particularly, this study seeks to compare the effect of pasteurization and non-pasteurization (i.e., a living bacterial culture) of a bacterial strain on the growth parameters of canola seedlings.

3-1/ Canola Cultivar

Brassica napus belonging to cultivar 5525CL (Brett Young) canola seedlings were disinfected according to the protocol described in Asaduzzaman et al. (“Metabolomics Differentiation of Canola Genotypes: Toward an Understanding of Canola Allelochemicals.” Frontiers in Plant Science, vol. 5, 2015.doi:10.3389/fpls.2014.00765). After drying, the seeds were left to germinate in Petri dishes containing agar (15 g/l). The germinated seeds were transferred into germination bags (Mega International), in an amount of 2 seeds/bag, containing a mixture of breeding ground supplemented with a half dose of Hoagland No. 2 (Sigma, H2395).

3-2/ Study Protocols

TABLE 2
Description of the various protocols studied in this example.

| Protocol No. | Protocols |
|---|---|
| 1 | Suspension of living Delftia acidovorans RAY 209 |
| | (cells washed in sterile distilled water) |
| 2 | Suspension of Delftia acidovorans RAY 209 |
| | (cells washed in sterile distilled water) |
| | inactivated by pasteurization |
| 3 | Suspension de Delftia acidovorans RAY 209 |
| | (cells washed in sterile distilled water) |
| | inactivated by French press treatment |
| | (high pressure followed by rapid decompression) |
| 4 | Suspension of living Lactobacillus rhamnosus R0011 |
| | (cells washed in sterile distilled water) |
| 5 | Suspension of Lactobacillus rhamnosus R0011 |
| | (cells washed in sterile distilled water) |
| | inactivated by pasteurization |
| 6 | Suspension of Lactobacillus rhamnosus R0011 |
| | (cells washed in sterile distilled water) |
| | inactivated by French press treatment |
| | (high pressure followed by rapid decompression) |
| 7 | Delftia acidovorans RAY 209 culture supernatant |
| | (treatment with no bacteria) |
| 8 | Sterile distilled water |

3-3 Preparation of Unpasteurized Inoculants

The bacterial strains used for the inoculation of canola seedlings are Delftia acidovorans RAY 209 (BBL) and Lactobacillus rhamnosus R0011 (Institut Rosell-Lallemand. Montréal, Qc, Canada). The concentrations of stock bacteria solutions are, respectively, 3.71E+08 CFU/ml and 2.21E+11 CFU/ml.

The stock solutions (1 ml) are centrifuged at 8500 rpm for 15 minutes. The pellet is resuspended in 1 ml of sterile distilled water. This bacterial cell washing step is repeated twice. Following the first centrifugation of the stock solution of Delftia acidovorans RAY 209, the supernatant is retained for the future treatment of canola seedlings (protocol 3). Bacterial suspensions in sterile distilled water are kept for the inoculation of germinated canola (protocols 1 and 4).

3-4/ Preparation of Inactivated Inoculants

Two inactivation techniques were tested: (1) pasteurization or heat treatment and (2) French press treatment (high pressure followed by rapid decompression).

(1) Heat Treatment

For each of the 2 bacterial strains studied, 1 ml of bacterial suspension at a concentration of 3.71E+08 CFU/ml in distilled water is transferred into an Eppendorf tube (1.5 ml) and incubated at 80° C. in a water bath for 20 minutes. The pasteurized bacterial suspensions are kept for the inoculation of germinated canola seeds (protocols 2 and 5).

(2) French Press Treatment (High Pressure Followed by Rapid Decompression)

For each of the 2 bacterial strains studied, 5 ml of bacterial suspension at a concentration of 2.21E+11 CFU/ml in sterile distilled water are put through the French press (American Instrument CO Inc.) at the pressure of 18000 psi at 4° C. followed by instantaneous depressurization. The lysed bacterial cells are recovered for the inoculation of germinated canola seeds (protocols 3 and 6).

3-5/ Bacterial Inoculation of Canola Seedlings

The germinated canola seeds were inoculated with a pipette and 10 μl of the preparations obtained (see Table 2 for the description of the 8 protocols studied) were applied onto each canola seed. Table 3 shows the bacterial concentrations applied onto the canola seeds.

The treated seeds were kept under controlled atmosphere in a growth chamber with 16 hours of light at 22° C. and 8 hours of darkness at 18° C.

The experimental setup included, for each protocol, 10 repetitions of 2 germinated seeds/germination bag. A total of 20 germinated seeds were therefore treated per protocol.

TABLE 3
Calculations of the bacterial concentrations applied per canola seed

| | | Stock | | | | | |
|---|---|---|---|---|---|---|---|
| | | solution | | | Final | Quantity of | Quantity of |
| | Stock | dilution to | Titer | Quantity | titer | inoculant | water |
| | solution | 10⁸ | required | applied/ | (CFU/seed | in the total | in the total |
| Inoculant | concentration | CFU/mL | (CFU/ml) | seed (μl) | (10 μl)) | volume (μL) | volume (μL) |
| D. acidovorans | 3.71E+08 | 3.71E+08 | 1.00E+08 | 10 | 1.00E+06 | 2.694 | 7.31 |
| RAY209 | | | | | | | |
| L. rhamnosus | 2.21E+11 | 2.21E+08 | 1.00E+08 | 10 | 1.00E+06 | 4.525 | 5.48 |
| R0011 | | | | | | | |

3-6/ Notation of Results

Data were taken after 8 days, 22 days and 36 days from inoculation of the canola seeds. After 8 days, the lateral roots were counted. After 22 days of incubation, 10 seedlings per protocol were harvested. Ten seedlings per protocol were harvested after 36 days of incubation. After harvesting the canola seedlings, the height of the shoots was measured from the collar to the highest leaf. The roots were separated from the soil and measured. The vegetal biomass and the root biomass (shoots and roots separately) were also measured using a precision balance. The dry weights of the shoots and roots were determined after drying at 35° C. for 48 hours.

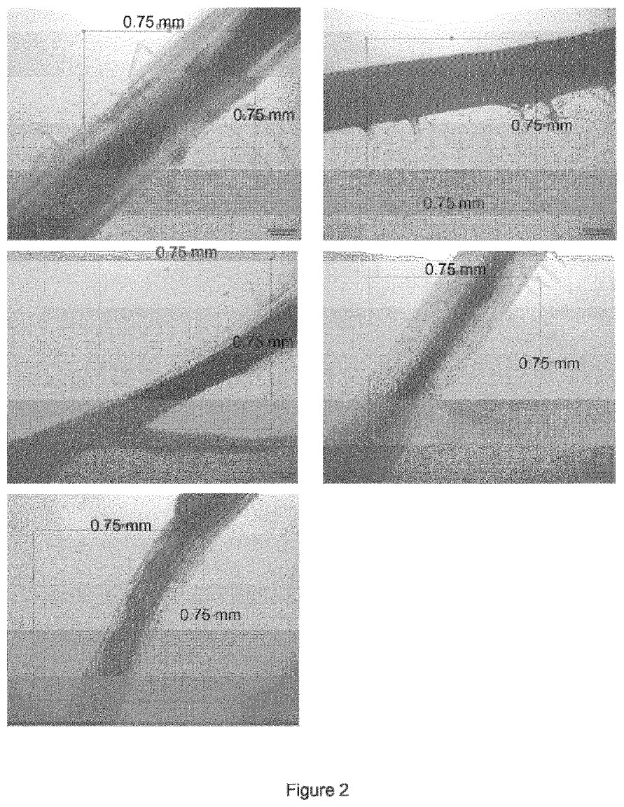

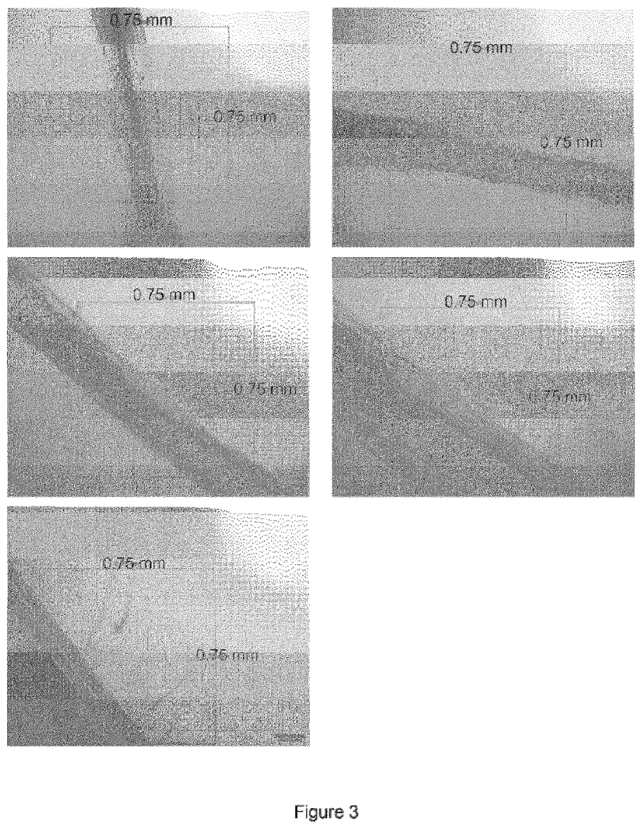

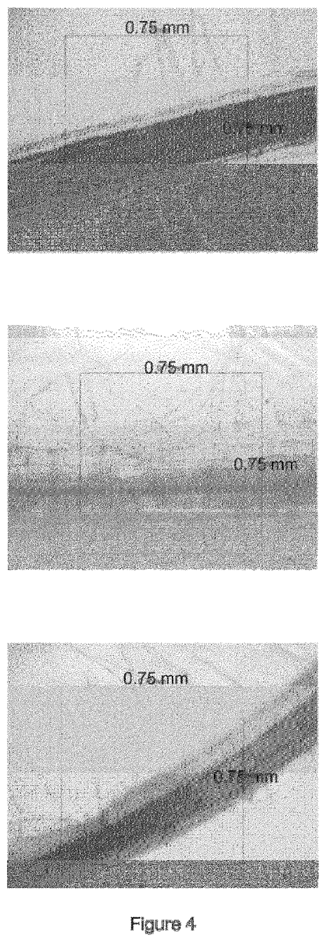

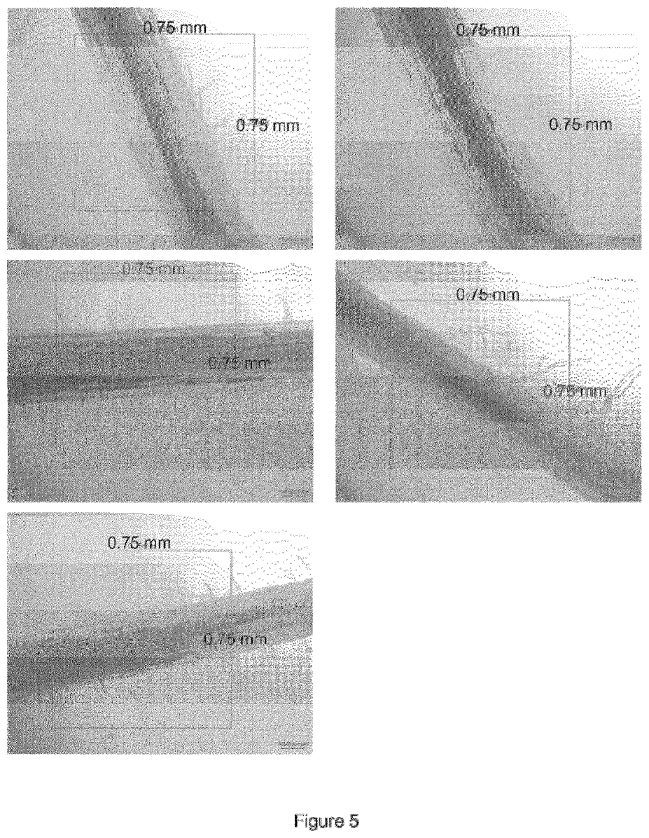

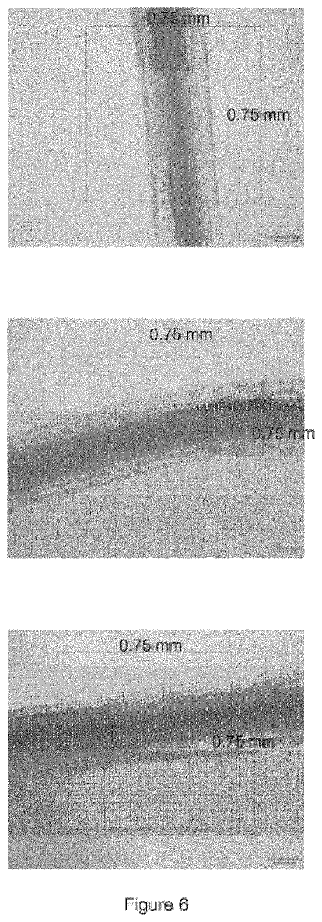

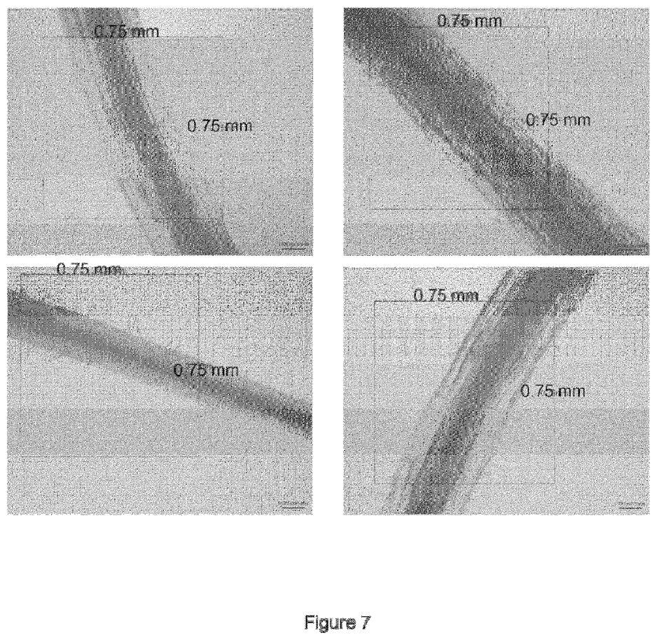

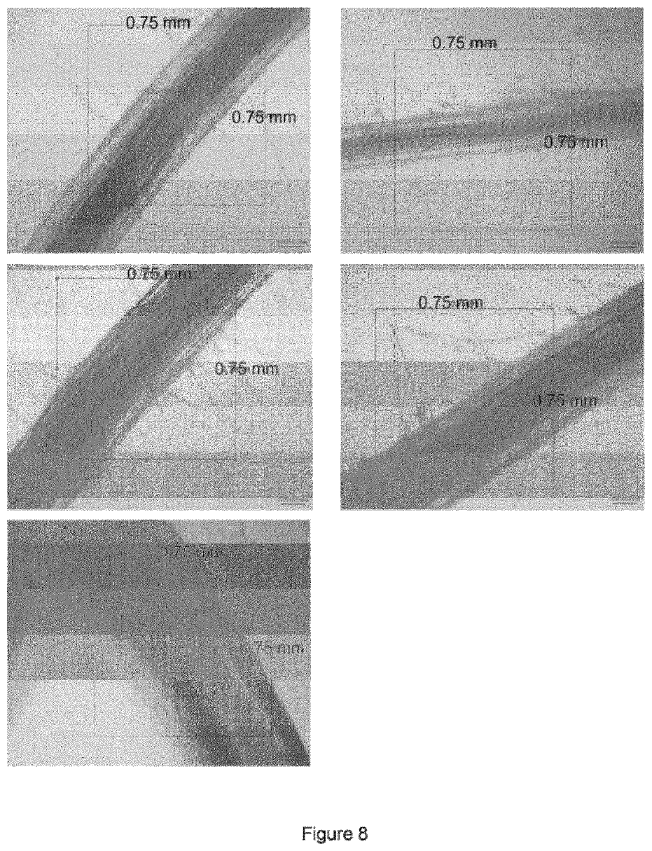

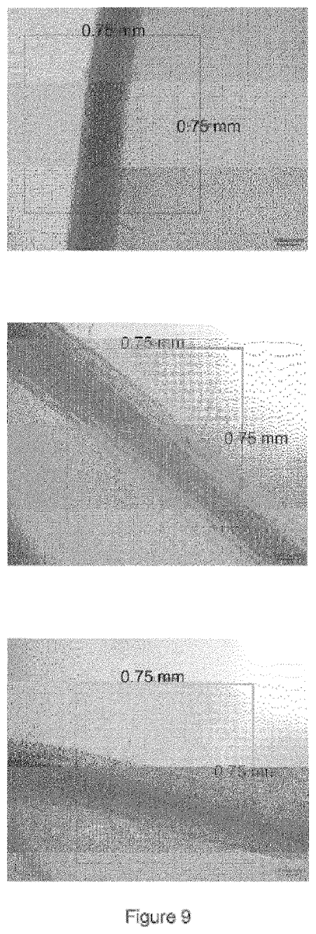

Moreover, for each of the protocols, the absorbant root hairs were observed under the microscope in order to evaluate their presence on the canola seedling roots. To do this, for each of the protocols, 1 cm of primary root was placed in a Petri dish. The root was immersed in water so that the absorbent root hairs were in suspension. The microscopic observations were done at an enlargement of 100×. The root hairs were counted over an area of 0.75 mm². FIGS. 2 to 9 show the images of microscopic observations of absorbant root hairs according to the protocol.

One-way analysis of variance (ANOVA) and Bartlett's variance comparison tests were performed, to conclude which protocols had a different variance from the others in shoot height (using KyPlot software). The result of the shoot height analysis is presented in Table 4.

The results show that the Delftia acidovorans strain can stimulate the development of the root part of the plants by increasing the number of absorbant root hairs. Among other things, this would have the effect of improving the assimilation of nutrients by the plants.

TABLE 4
Comparison of the height of canola shoots 36 days after treatment.

| | Sterile | Living |
|---|---|---|
| Treatment | water | Delftia |
| Sterile water | X | X |
| Delftia culture medium | + | X |
| Lactobacillus inactivated by pressure | − | X |
| Pasteurized Lactobacillus | + | X |
| Living Lactobacillus | − | X |
| Living Delftia | −− | X |
| Delftia inactivated by pressure | + | ++ |
| Pasteurized Delftia | +++ | +++ |

The symbols represent the significance of the results from the comparison of the treatments indicated in the column relative to the treatments indicated in the line (ANOVA analysis of variance statistical test).
Legend
symbol meaning
“+” no significant positive difference
“−” no significant negative difference
“=” no differences
“++” significant positive difference at P = 0.1
“−−” significant negative difference at P = 0.1
“+++” significant positive difference at P = 0.05
“−−−” significant negative difference at P = 0.05

Although the present invention has been described with the aid of specific implementations, it is understood that several variations and modifications can be added to said implementations, and the present application aims to cover such modifications, uses or adaptations of the present application in general, the principles of the invention and including any variation of the present description which will become known or conventional in the field of activity in which the present application is found, and which can be applied to the essential elements mentioned above, in accordance with the scope of the claims.

1. A method for increasing the biostimulant efficacy on plant development of a living bacteria strain or a composition containing it, the method comprising a step of inactivating the living bacteria strain, said inactivated strain thus obtained having a greater biostimulant efficacy on plant development than that obtained with the same strain of living bacteria or with a composition containing it.

2. The method according to claim 1, wherein the living bacteria strain is inactivated by physical, biochemical, chemical or physicochemical processes.

3. The method according to claim 1, wherein the living bacteria strain is inactivated by heat or high pressure treatment.

4. The method according to claim 1, wherein the living bacteria strain is inactivated by pasteurization.

5. The method according to claim 1, wherein the living bacteria strain is inactivated without its culture medium.

6. The method according to claim 1, wherein the bacteria strain is of the genus Delftia, Achromobacter, Agrobacterium, or Stenotrophomonas.

7. The method according to claim 6, wherein the bacteria strain is of the genus Delftia.

8. The method according to claim 6, wherein the bacteria strain is Delftia acidovorans RAY209 filed with the ATCC on Apr. 25, 2002 under no. PTA-4249, Achromobacter piechaudii RAY12 filed with the American Type Culture Collection (ATCC) on Apr. 16, 2002 under no. PTA-4231, Agrobacterium tumefaciens RAY28 filed with the American Type Culture Collection (ATCC) on Apr. 16, 2002 under no. PTA-4232, or Stenotrophomonas maltophilia RAY 132 filed with the American Type Culture Collection (ATCC) on Apr. 16, 2002 under no. PTA-4233.

9. The method according to claim 8, wherein the inactivated bacteria strain is the Delftia acidovorans strain RAY209 filed with the ATCC on Apr. 25, 2002 under no. PTA-4249.

10. An inactivated bacteria strain to improve plant development, wherein the inactivated bacteria strain has a greater biostimulant efficacy on plant development than that obtained with the same living bacteria.

11. The inactivated bacteria strain according to claim 10, wherein the inactivated bacterial strain is of the genus Delftia, Achromobacter, Agrobacterium, or Stenotrophomonas.

12. The inactivated bacteria strain according to claim 11, wherein the inactivated bacterial strain is of the genus Delftia.

13. The inactivated bacteria strain according to claim 11, wherein the inactivated bacteria strain is chosen from among the group consisting in the strain Delftia acidovorans RAY209 filed with the ATCC on Apr. 25, 2002 under no. PTA-4249, Achromobacter piechaudii RAY12 filed with the American Type Culture Collection (ATCC) on Apr. 16, 2002 under no. PTA-4231, Agrobacterium tumefaciens RAY28 filed with the American Type Culture Collection (ATCC) on Apr. 16, 2002 under no. PTA-4232, or Stenotrophomonas maltophilia RAY 132 filed with the American Type Culture Collection (ATCC) on Apr. 16, 2002 under no. PTA-4233.

14. The inactivated bacteria strain according to claim 13, wherein the inactivated bacteria strain is the Delftia acidovorans strain RAY209 filed with the ATCC on Apr. 25, 2002 under no. PTA-4249.

15. The inactivated bacteria strain according to claim 10, characterized in that it is inactivated by a physical, chemical, biochemical or physicochemical process.

16. The inactivated bacteria strain according to claim 10, wherein the strain is inactivated by thermal treatment or high pressure treatment.

17. The inactivated bacteria strain according to claim 10, wherein the strain is inactivated by pasteurization.

18. A composition to improve plant development, further comprising at least one inactivated bacteria strain according to claim 10.

19. The composition to improve plant development according to claim 18, further comprising at least one inactivated bacteria strain and a vehicle compatible for agriculture.

20. The composition according to claim 18, further comprising an inactivated bacteria strain isolated from its culture medium.

21. The composition according to claim 18, further comprising the inactivated bacteria strain in combination with other living microorganisms, inactivated or in extracts.

22. The composition according to claim 21, further comprising the inactivated bacteria strain in combination with other inactivated bacteria strains.

23. The composition according to claim 18, further comprising fertilizer, herbicides, insecticides, fungicides, mineral solutions and/or growing media.

24. The composition according to claim 18, wherein the composition is present in an appropriate form for soil treatment, treatment of the root part of the plant, treatment of the leaf part of the plant, treatment of the flowering parts, treatment of the fruiting parts and/or treatment of the seed.

25. The composition according to claim 18, wherein the composition is present in the form of powder, granules, microgranules, seed treatments, liquid formulations, bacteria encapsulations or liquid suspensions.

26. (canceled)

27. A method to improve plant development comprising the administration of an effective amount of a composition according to claim 18.

28. A plant obtained according to the method of claim 27.
